(19) United States
(12) Patent Application Publication
(10) Pub. No.: US 2007/0173801 A1
(43) Pub. Date: Jul. 26, 2007

(54) SPINE IRRITATION RELIEF AND DEGENERATION AVOIDANCE/REVERSAL APPARATUS AND METHOD

(75) Inventor: John A. HIPP, Manvel, TX (US)

Correspondence Address: CONLEY ROSE, P.C.;David A. Rose, P. O. BOX 3267, HOUSTON, TX, 77253-3267, US

(73) Assignee: BAYLOR COLLEGE OF MEDICINE, Houston, TX (US)

(21) Appl. No.: 11/460,389

(22) Filed: Jul. 27, 2006

Related U.S. Application Data
(60) Provisional application No. 60/595,695, filed on Jul. 28, 2005.

(51) Int. Cl. A61B 18/04

(52) U.S. Cl. 606/33

(57) ABSTRACT

A system and a method for the relief of pain/irritation of the spine or for the prevention of degeneration of the spine. The system is designed to facilitate creep in the human spine in a way that can be self-administered and with which patients will readily comply. The apparatus comprises a base, an inclinable support surface pivotably connected to the base, said support surface capable of supporting the upper body of the person in a prone position, a support member coupled to the base and the support surface, and an actuator coupled to the support member for controlling the angle of inclination of the support surface to promote distraction of the spine. The method comprises positioning a person on the support surface and adjusting the angle of inclination thereof so that the device supports the upper body of the person and gravity causes distraction of the spine.

### CROSS-REFERENCE TO RELATED APPLICATIONS

The present application claims the benefit under 35 U.S.C. §119(e) of U.S. Provisional Application No. 60/595695 filed Jul. 28, 2005 and entitled “Spine Irritation Relief Apparatus”, which is hereby incorporated herein by reference for all purposes.

### STATEMENT REGARDING FEDERALLY SPONSORED RESEARCH OR DEVELOPMENT

Not Applicable

### Field of the Invention

The present invention relates generally to an apparatus and method for relieving irritation of the spine and the avoidance and/or reversal of spine degenerations

### BACKGROUND OF THE INVENTION

Back pain is an enormous burden to society, with almost 80 percent of people suffering from back pain at some point in their lives. Back pain accounts for more lost work days than any other medical condition and it is estimated that the incremental cost of back pain to society was almost $27 billion in 2002. The incidence and impact of back pain in children and young adults is also increasing.

Reducing the impact of back pain on society has proven elusive. By way of illustration, a Medline search for the phrase “low back pain” results in over 10,000 references from 1950 to the present, and addition of the term “treatment” reduces this number to 6,461. Despite these thousands of research studies that may be addressing the problem of back pain, there is little clear evidence that the typical back pain patient is likely to fare any better today than they would have if they were treated a decade or more ago. The only evidence of a global change in the impact of back pain to society was in an NIH (National Institutes of Health) progress report suggesting that activity limitations due to chronic back pain have declined from 32 to 26 percent from 1997 to 2002, although an explanation for this apparent decline was not provided (based on data from the National Health Interview Survey of the CDC, as reported in the 2002 progress report for the Healthy People 2010 program of the Department of Health and Human Services, DHHS). The DHHS has included reducing the cost of back pain as a specific objective in their Healthy People 2010 program, although since this program was started in 2000, no major milestones have been reported.

There are now many different surgical and non-surgical treatments that are offered to patients with back pain and related symptoms. There are many more treatments that are in development or clinical trials. The existing scientific evidence suggests that many of the surgical treatment options can provide some benefit to some patients. However, it is also clear that improperly selected patients will do worse with many of these treatments. Unfortunately, validated patient selection guidelines that are proven to be reliable and efficacious are not available for any current surgical option. For this reason, and because the least invasive treatment is, in most instances, preferable for patients, non-surgical treatment options are almost always the first line of treatment for patients with low back pain.

There exist many non-surgical treatment options for patients experiencing back pain. Avoiding certain activities and using mild anti-inflammatory and pain medication is frequently the first treatment offered. Physical therapy, massage, chiropractic manipulations, and heat or cold therapy are other commonly offered treatments, although systematic reviews of many of these options have generally concluded that most of these treatments are not based on rigorous scientific evidence. Numerous treatment guidelines have also been published, but the scientific basis for many of these guidelines is poor, and the clinical efficacy of the guidelines has never been established.

During the degenerative process that occurs in the lower spine of many people as they age and accumulate mechanical damage to the spine, the intervertebral discs lose height and elasticity. The discs are thereby less able to absorb the mechanical loads that occur during activities of daily living. In addition, loss of disc height is associated with an increasing percent of loads being transferred through the facet joints. As the facet joints support higher loads, the cartilage degrades and osteophytes begin to form around the periphery of the joints. In addition, osteophytes can form along the edges on the endplates of the vertebrae. The loss of disc height and the concomitant enlargement of the facets and endplates together reduce the amount of space available for the blood vessels and nerve tissue that passes through the spinal canal and the neural foramen. This is believed to result in mechanical irritation of the nerve and vascular structures. Numerous studies have shown that mechanical compression of nerve roots, particularly if the regional tissue is inflamed, results in pain. It is also well-known that surgical decompression of the spinal canal and neural foramen will alleviate symptoms in many patients. These surgical decompression procedures are, however, not without complications. A non-surgical method for decompressing the spinal canal and nerve roots may similarly provide relief of back pain and related symptoms.

It is known that the intervertebral discs increase in height after sleep. Multiple studies have demonstrated that unloading the spine can increase disc height and improve the mechanical behavior of the spine. The mechanism by which the spine changes during periods of relatively low loading is complex, but is known to involve the process of mechanical creep. Mechanical creep is the elongation that occurs over time in a material or structure when subject to a constant sustained load. This process of creep can be facilitated by subjecting the spine to periods of low loads, but even more so with application of a sustained distractive load. Subjecting the spine to distractive loads over an extended period of time, and doing this frequently enough to encourage healing and perhaps regeneration of any inflamed or damaged tissues in and around the spine is challenging.

### SUMMARY OF THE INVENTION

A specially designed mechanical system and method of using said system has been developed to treat and prevent recurrence of lower back pain and related symptoms. The system is designed to facilitate creep in the human spine in a way that can be self-administered and with which patients will readily comply. This system consists of a soft surface, designed to hold a person in a prone position on an incline that is very comfortable and prevents the upper body from slipping down the inclined surface. In this disclosure, “prone” is used to imply a position with the front of the body turned toward the supporting surface. The supporting surface may be inclined or fully horizontal, but the word “prone” as used herein does not require that the body be fully horizontal.

The intention of the system is to relax the body and allow gravitational forces to create distraction on the lumbar spine through the weight of the lower body, causing the lower body to slide down the inclined surface away from the upper body, which is prevented from sliding down the surface. Preliminary studies show that this action facilitates mechanical creep in the ligaments, intervertebral disc and muscles spanning between vertebrae. The desire is to increase the height, hydration, and nutrition of the tissues within the intervertebral disc and facet joints, and also to increase the volume available for the containment of the nerves and vascular components within the spinal canal and neural foramen. Maintaining the body on an inclined surface may also allow for drainage of venous blood from bone and tissues that comprise the spine, and this may also reduce excitation of pain sensing nerves within the bone and tissues of the spine.

Since the hypothesized mechanisms and intended goals of this system likely require that the patient spend an hour or more lying continuously on the inclined surface, in order to give the spine time to heal, the surface is also designed to allow patients to work on a computer, read, watch television, listen to music, attend meetings, talk on a telephone, and otherwise distract themselves while also beneficially distracting their spine.

In addition to positioning a person in a way that can facilitate healing of tissues that are causing back related symptoms, there is also scientific evidence supporting that the described invention may also help prevent or reverse degenerative changes in the spine. Therefore, the described invention may not only be useful to people presenting with back-related symptoms, it may also be of value to people who would like to avoid degenerative changes in the spine.

This apparatus could be used in private homes, public venues, such as theaters, coffee houses, etc., and may even prove valuable as an operating table.

Disclosed herein is an apparatus comprising a base, an inclinable support surface pivotably connected to said base, said support surface capable of supporting the upper body of the person in a prone position, a support member coupled to said base and said support surface, and an actuator coupled to said support member operable for controlling the angle of inclination of said support surface.

In an embodiment the apparatus comprises a lower body support surface and a torso immobilizer coupled to the support surface to secure the upper body of a person on the support surface when the support surface is tilted up or down. In an embodiment, the torso immobilizer has an open position and a closed position and encloses at least a portion of the upper body of a person when the torso immobilizer is in the closed position. In an embodiment, the torso immobilizer comprises a pivoted rigid support structure comprising a pair of arms, armpit supports attached to the pivoted rigid support structure, and a torso contact surface attached to the pivoted rigid support structure.

In an embodiment, the support surface of the apparatus is a contoured support surface. In an embodiment, the support surface of the apparatus is a contoured support surface that is contoured to accommodate the body shape of the person. In an embodiment, the support surface of the apparatus is a contoured support surface that is custom-made to accommodate the body shape of the person.

Also disclosed herein is a method for relieving and preventing irritation to the spine of a person. The method comprises (a) providing an apparatus having an inclinable support surface adapted for supporting the body of a person in a prone position and an actuator that controls the angle of inclination of the support surface; (b) positioning the person on the support surface, wherein the torso and at least a portion of the lower body of the person are supported by the support surface and (c) adjusting the angle of inclination of said support surface after positioning said person on said support so that there is adequate distraction of the spine.

The present invention comprises a combination of features and advantages which enable it to overcome various problems of prior devices and methods. The various characteristics described above, as well as other features, will be readily apparent to those skilled in the art upon reading the following detailed description of the preferred embodiments of the invention, and by referring to the accompanying drawings.

### BRIEF DESCRIPTION OF THE DRAWINGS

For a more detailed description of the preferred embodiment of the present invention, reference will now be made to the accompanying drawings, wherein:

FIG. 1 is a side view of a spine distraction device constructed in accordance with a first embodiment of the present invention;

FIG. 2 is a side view of a person engaging the device of FIG. 1;

FIG. 3 is a side view of a person engaging the device of FIG. 1 and having assumed a partially prostrate position; and

FIG. 4 is a front view of the device of FIG. 1;

FIG. 5 is a side view of a person engaging a device constructed in accordance with a second embodiment of the present invention;

FIG. 6 is a front view of the device of FIG. 5;

FIG. 7 is a schematic cross-section illustrating the operation of the torso support member the device of FIG. 5;

FIG. 8 is a partial side showing a person engaging the head support of a third embodiment of the present invention; and

FIG. 9 is an enlarged partial view illustrating one embodiment of a connection for the head support of FIG. 8.

### DETAILED DESCRIPTION OF THE PREFERRED EMBODIMENTS

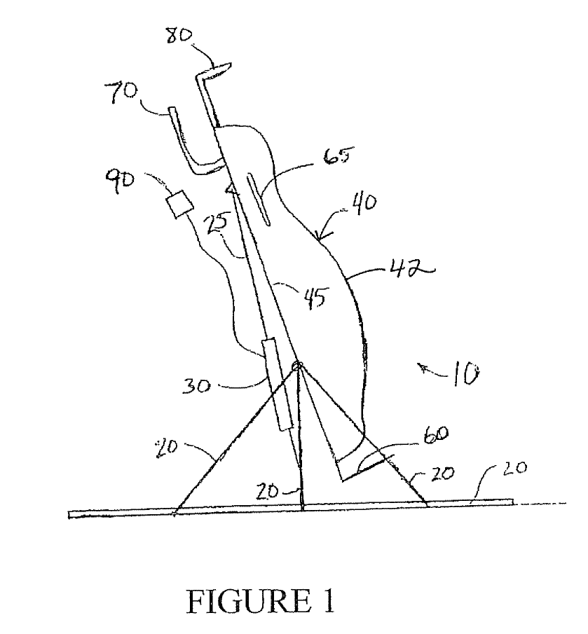

In the embodiment shown in FIGS. 1-4, a spine irritation relief apparatus (SIRA) 10 comprises a base 20, a support member 25, a body support 40 having a support surface 42, lower surface 45, and an actuator 30 coupled to support member 25 and base 20. In the embodiments shown in FIGS. 1-4, SIRA 10 comprises a foot support 60, a pair of hand grips 65, a peripheral support 70, a head support 80, and a controller 90. In this embodiment, support surface 42 is a contoured support surface. Lower surface 45 and contoured support surface 42 may or may not be a single unit. Other embodiments of the present invention may not incorporate all of the above-mentioned features.

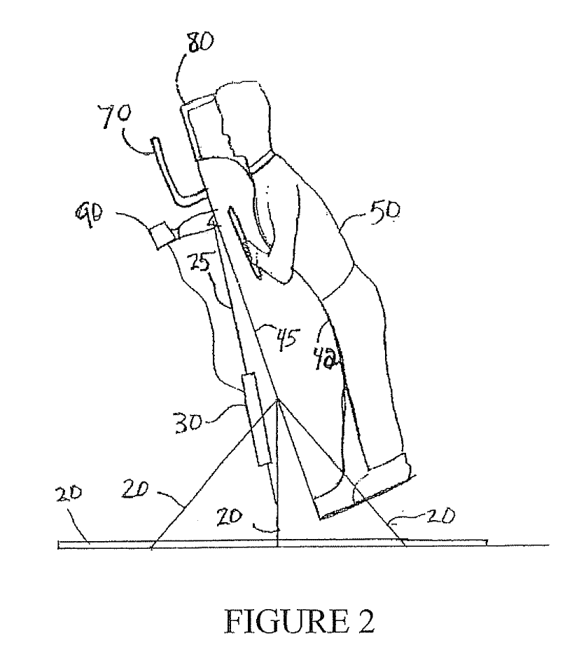

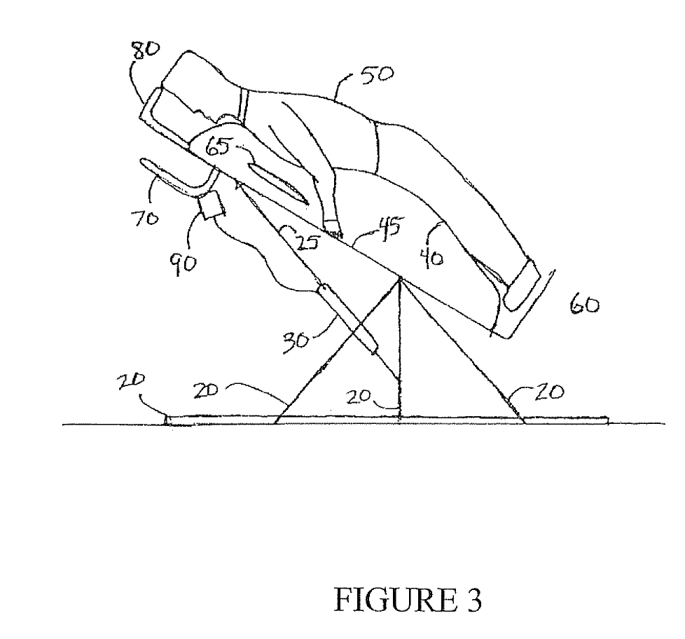

As shown in FIG. 2, a person 50 preferably first engages SIRA 10 when contoured support surface 40 is in a near-vertical position. Person 50 can use foot support 60 and/or hand grips 65 to help position him- or herself relative to contoured support surface 40. After such positioning, person 50 leans forward so that contoured support surface 40 contacts the front of person 50. Person 50 may then use controller 90 to control actuator 30, causing support member 25 to retract, thereby changing the position of body support 40. Actuator 30 may be an electric, hydraulic, or other type of actuator suitable for controlled movement. By changing the position of support member 25, body support surface 40 can be rotated to a more or less prostrate position, as shown in FIGS. 2 and 3. In an alternative method of use, person 50 can use controller 90 to rotate body support 40 to a more horizontal position than that shown in FIG. 2 before engaging the device. This method may be particularly preferred in embodiments of the present invention that do not comprise a foot support 60 or hand grips 65.

In FIG. 3, person 50 has used controller 90 and actuator 30 to rotate body support 40 so that the angle between support 40 and horizontal is decreased. However, as shown in FIG. 3, support 40 is still somewhat inclined with respect to horizontal. Many users of the device will find that an angle of twenty-five to forty-five degrees from horizontal provides an optimal effect. This allows the weight of the lower body of person 50 to create tensile forces on the spine of person 50. If necessary, the position of foot support 60 may be adjusted or automatically shifted in certain embodiments so that the weight of the lower body is not supported by foot support 60 after contoured support surface 40 has been rotated. As the spine of person 50 is subjected to tensile forces, mechanical creep is induced in the spine, which can relieve irritation and reduce pain associated with the spine and help prevent or reverse degeneration of the spine.

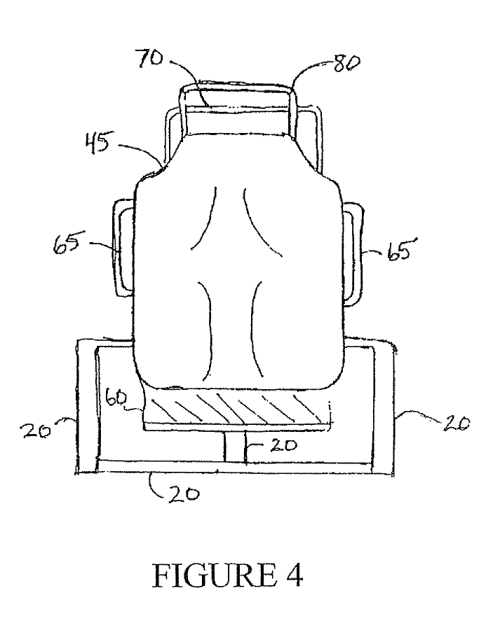

In FIG. 4, a front view of one embodiment of SIRA 10 is shown. From this view, the head support 80, peripheral support 70, hand grips 65, and foot support 60 can be more clearly seen. As shown, hand grips 65 and foot support 60 can be used to allow a person to easily position him or herself in front of body support 40. Head support 80 allows person 50 to comfortably rest his or her head so that person 50 may comfortably use SIRA 10 for extended periods of time. Peripheral support 70 can include attachments that allow it to support numerous devices, such as a telephone, a book, a keyboard, a computer mouse, a computer display, a television screen, a workspace, a writing surface, a controller for an entertainment system, a drink holder, a food tray, or other devices (not shown). The ability to access such peripheral devices allows person 50 to remain productive and engage in other activities while using SIRA 10. This increases the likelihood that person 50 will use SIRA 10 for an extended period of time, and therefore will increase the chances that SIRA 10 will provide effective relief to and prevent degeneration of the spine of person 50.

Referring back to FIG. 3, it can be seen that the contoured support surface 42 provides a comfortable surface upon which person 50 lies while using SIRA 10. Contoured support surface 42 can be designed to specifically match the dimensions of a particular person 50. This can be accomplished in various methods, such as laser scanning the front surface of person 50 and manufacturing contoured support surface 42 based on the dimensions obtained from the scan. Other methods of manufacture may include techniques such as impression molding, in which person 50 lies on a soft material that can be used to create a mold for contoured support surface 42. In addition, a different contoured support surface 42 can be designed to accommodate either men or women or can be designed for a range of differently-sized persons 50. In an embodiment, the contoured support surface may be interchangeably connected to the base to allow a person to use a contoured support surface designed for his body.

In addition to increasing the comfort of person 50, contoured support surface 40 also helps prevent the upper body of person 50 from sliding down while the device is in use. In certain embodiments, surface 42 is covered with a material selected such that the coefficient of friction between contoured support surface 42 and natural fiber clothing is greater than 0.6. In other embodiments, the contoured support surface 42 may have different coefficients of friction in the portions which support the upper torso and the lower body. For example, the coefficient of friction in the upper portion may be higher than the coefficient of friction in the lower portion, allowing the lower portion to subject the spine of person 50 to an increased tensile load. As shown in FIG. 3, when the lower body follows the contours of contoured support member 40, it causes the back of person 50 to be extended or stretched. In the embodiment of FIG. 3, controller 90 can be connected to the peripheral support 70. This allows person 50 to easily access controller 90 and adjust the angle of contoured support member 40 to the desired position.

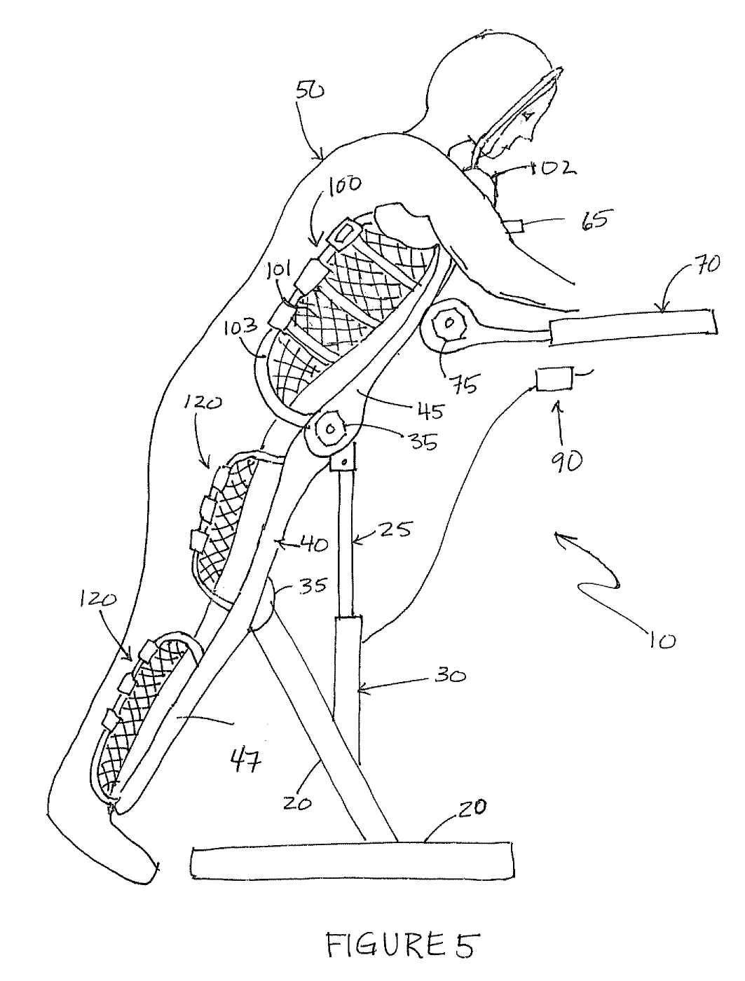

As shown in FIGS. 5-8, a second embodiment of the present invention incorporates a collapsible hammock-type torso immobilizer 100. As shown in FIG. 5, this embodiment includes a base 20, support member 25, body support 40, hammock-type or upholstered (not shown) lower body supports 120, upper body support member 45, lower body support member 47, an actuator 30 coupled to support member 25 and base 20, hand grips 65, peripheral support 70, head support 80, controller 90, torso immobilizer 100, a pivots 35 for adjusting the relative positions of upper body support member 45 and lower body support member 47, and pivot(s) 75 for adjusting the position of peripheral device 70. Other embodiments of the present invention may not incorporate all of the above-mentioned features.

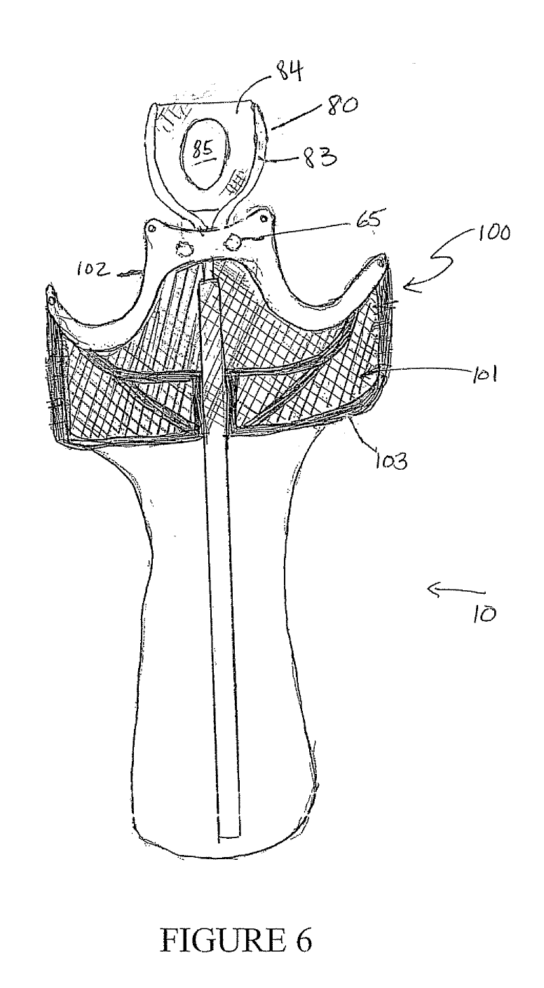

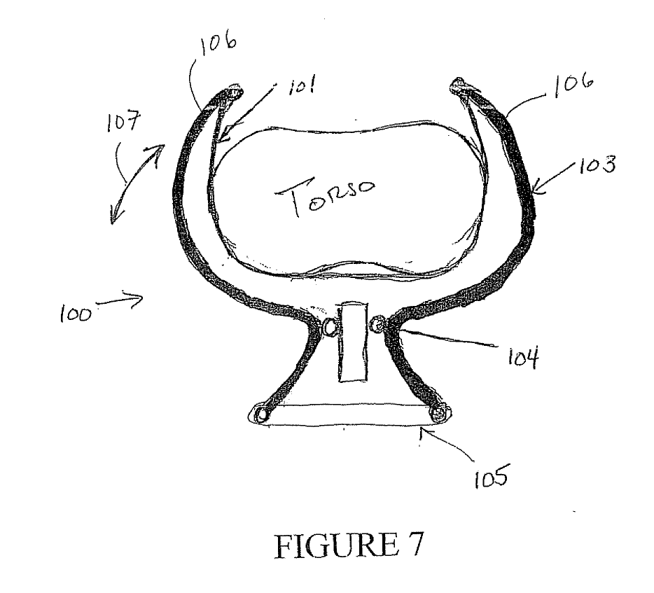

In this second embodiment, the upper body is held against the apparatus 10 through the use of torso immobilizer 100, which is shown in more detail in FIGS. 6 and 7. Torso immobilizer 100 preferably includes a mesh hammock-type inner support layer 101, armpit supports 102, rigid peripheral support structure 103, pivots 104, and at least one elastic strap 105. As seen in FIG. 7, the rigid support structure 103 includes a pair of arms 106 that each pivot around a respective pivot 104, as indicated by arrow 107. The lower portion of each arm 106 curves away from the pivot point 104 and at its remote end is retained by elastic strap 105. Thus, elastic strap 105 tends to urge the lower ends of the arms together, which in turn causes the upper ends of the arms to open. It will be understood that elastic strap 105 can be replaced with any other suitable biasing means. When the device is not in use, the torso immobilizer is held open by this mechanism, making it easy for a user to enter and engage the device.

As best seen in FIG. 6, a mesh or netting 101 extends between the upper portions of arms 106. Mesh 101 is preferably but not necessarily an open-weave mesh that is at least somewhat elastic. When the device is in use, the weight of the user tends to bring the upper ends of arms 106 together, against the biasing action of elastic means 105. As arms 106 come together, mesh 101 partially encircles the torso of the user, as shown in FIG. 7. This results in a comfortable and secure support of the user's body.

Armpit supports 102 (most clearly seen in FIGS. 5 and 6) preferably comprises a fabric-wrapped foam that is designed to comfortably support the armpits of person 50 when they are supported in the device. Optional hand grips 65 may be, for example, plastic and may be attached to the foam of the armpit support 102 or, alternatively, to the lower surface 45 of the apparatus 10. Likewise, a head support 80 may extend from upper body support member 45 or another part of the device.

In the embodiment of FIGS. 5-7, a person 50 advances to the apparatus 10 when it is in an upright or nearly upright position, places his arms over the contoured armpit support 102 and, by bending his arms, optionally grasps hand grips 65. Using controller 90, which controls the preferably continuously-adjustable electric actuator 30, person 50 adjusts the inclination of apparatus 10 to the desired value. As the person's weight shifts forward onto the device, the two sides of the torso immobilizer pivot about pivots 104 and partially close about the torso of the person 50. The degree to which the arms of the immobilizer close is controlled by the length/elasticity of the elastic straps 105. The mesh webbing 101 between the arms forms a comfortable torso contact surface, as it stretches to some degree to fit the body type of the human 50 utilizing the apparatus, and due to the incorporation of elastic straps 105, which may be tightened or loosened, the apparatus incorporating the torso immobilizer is essentially a one-size-fits-all unit. This pivotable net and frame design allows person 50 to be supported from the back and sides as well as from the front, thus spreading the load and forming a comfortable immobilization system. The mesh also allows for air circulation to cool the person 50, which may be preferred when the apparatus is to be used in warmer climates and for longer durations, such as for naps or overnight sleeping. The lower-body supports 120 are preferably similar, non-pivotable hammock-type supports, but may also comprise a continuous upholstered padded surface that may be flat or contoured and may or may not leave the knee caps unsupported. In an alternative embodiment, a vest, coupled to the support surface, may grip the upper body of a person to hold the person on the support surface.

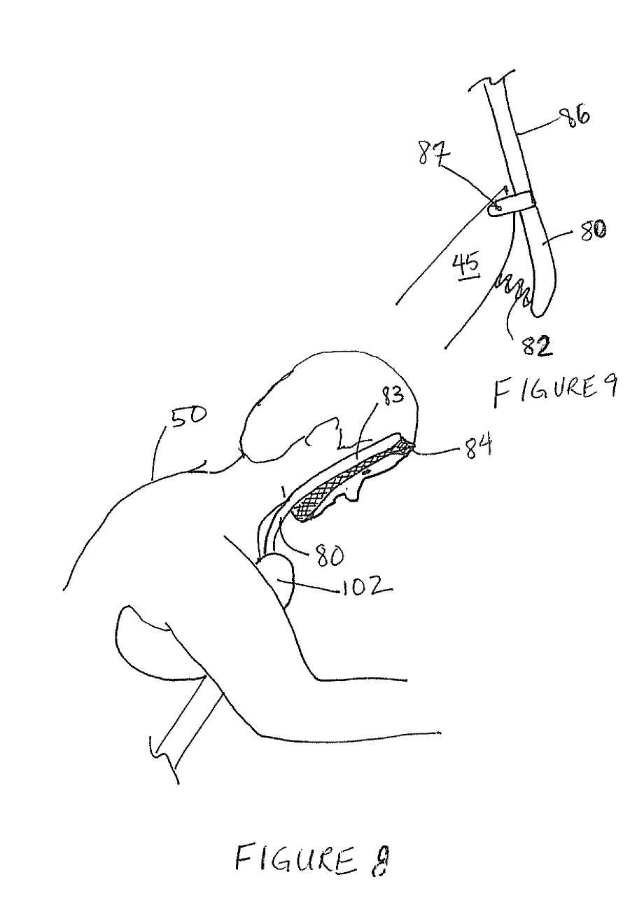

As shown in FIGS. 6 and 8, head support 80 preferably comprises a generally circular frame 83 that supports a flexible face support 84. Face support 84 preferably comprises a web having a round or oval 85 opening therein. The web extends across frame 83 such that when the device is inclined the web receives and comfortably supports his face. To that end face support 84 preferably comprises a fabric, such as for example, washable spandex. This would allow for the maintenance of a clean facial contact region and for the use of a person's own facial support material if he were to use an apparatus 10 that was available at a theater, for example.

Head support 80 is preferably pivotably mounted on the upper end of upper body support 45. According to the embodiment shown in FIG. 9, head support 80 is secured to support 45 by a pivotable bracket 87 and the lower end 86 of head support 80 engages a spring or other biasing means 82. Biasing means 82 resists movement of lower end 86 toward support 45, thereby also resisting forward movement of the face support. Further, head support 80 is preferably removable from the SIRA.

As in the embodiment of FIGS. 1-4, the peripheral support 70 is shown in FIG. 5. Peripheral support 70 preferably pivots about pivot(s) 75 and thus may be adjusted to the desired position or moved out of the way if so desired.

In any embodiment of the present invention, the weight of the lower body of person 50 is used to create tensile forces on the spine of person 50. When the spine of person 50 is subjected to tensile forces, a mechanical creep is induced in the spine which can relieve irritation and reduce pain associated with the spine, as described hereinabove. When contoured support member 40 is inclined more toward vertical, the amount of tensile force exerted on the spine is increased. Conversely, as contoured support member 40 is rotated more towards horizontal, the amount of tensile force exerted on the spine is decreased. The user can operate controller 90 to adjust the angle of inclination until the desired amount of tension is placed on the spine. The angle of inclination may be from about 0 to about 90 degrees, alternatively from about 30 to about 90 degrees, and alternatively about 60 degrees. The use of SIRA 10 provides a simple method of relieving irritation and/or preventing degeneration of the spine and, hence, may be used to avoid much more costly and complex procedures to reduce and/or prevent spinal irritation.

While preferred embodiments of this invention have been shown and described, modifications thereof can be made by one skilled in the art without departing from the scope or teaching of this invention. The embodiments described herein are exemplary only and are not limiting. Many variations and modifications of the system and apparatus are possible and are within the scope of the invention such that the relative dimensions of various parts, the materials from which the various parts are made, and other parameters can be varied. For instance, the peripheral device support may comprise a tray or shelf or other support mechanism that is hinged or otherwise articulated to allow a user to position the support to a location most comfortable for the user. Like wise, the various components of the device can be constructed from other materials and in configurations other than those shown and described herein. In addition, use of the terms “between”, “coupled to” or “connected” (and variations thereof) when describing the location of a component should not be construed such that the component must be directly contacting the adjacent components. Accordingly, the scope of protection is not limited to the embodiments described herein, but is only limited by the claims that follow, the scope of which shall include all equivalents of the subject matter of the claims.

What is claimed is:

1. An apparatus for relieving and preventing irritation of the spine of a person, comprising:
a base;
an inclinable support surface pivotably connected to said base, said support surface being capable of supporting the upper body of the person in a prone position;
a support member coupled to said base and said support surface; and
an actuator coupled to said support member, wherein said actuator is operable to adjust the angle of inclination of said support surface.

2. The apparatus of claim 1 wherein said support surface comprises:
a torso immobilizer coupled to said support surface, said torso immobilizer secures the upper body of said person on said support surface when said support surface is tilted up or down,
wherein said torso immobilizer has an open position and a closed position, said torso immobilizer encloses at least a portion of the upper body of said person in said closed position; and
a lower body support surface.

3. The apparatus of claim 2 wherein said torso immobilizer comprises:
a pivotably mounted rigid support structure comprising a pair of arms, said rigid support structure including a pair of armpit supports and a torso supporting means.

4. The apparatus of claim 3 wherein said torso immobilizer further comprises at least one biasing means attached to said rigid support structure such that said biasing means controls the degree to which said arms of said torso immobilizer close.

5. The apparatus of claim 2, further comprising a pair of hand grips attached to said torso immobilizer.

6. The apparatus of claim 1 wherein said support surface is capable of supporting the upper torso of the person and at least a portion of the lower body of the person when the person lies on said support surface.

7. The apparatus of claim 1 wherein the coefficient of friction between said contoured support surface and a natural fiber material is sufficient to prevent the upper torso of said person from sliding down said contoured support surface when said contoured support surface is placed at an angle between approximately twenty-five degrees and approximately forty-five degrees from horizontal.

8. The apparatus of claim 7 wherein the coefficient of friction between said contoured support surface and said natural fiber material is greater than 0.6.

9. The apparatus of claim 1, further comprising a foot support coupled to said support surface.

10. The apparatus of claim 1, further comprising a head support coupled to said support surface.

11. The apparatus of claim 10 wherein said head support is adjustable via a spring or pivot.

12. The apparatus of claim 1, further comprising a support for a peripheral device attached to said support surface.

13. The apparatus of claim 1 wherein the angle of inclination of said support surface is adjustable between about 0 degrees and about 90 degrees relative to horizontal.

14. The apparatus of claim 1, further comprising a vest that grips the upper body of said person and attaches to the support surface to help hold the upper-body of said person on the support surface.

15. A method for relieving and preventing irritation of the spine of a person comprising:
(a) providing an apparatus having an inclinable support surface adapted for supporting the body of said person in a prone position and an actuator that controls the angle of inclination of said support surface;
(b) positioning said person on said support surface, wherein the torso and at least a portion of the lower body of said person are supported by said support surface; and
(c) adjusting the angle of inclination of said support surface after positioning said person on said support surface so as to cause gravity to extend the spine so as to relieve irritation of the spine of said person.

16. The method of claim 15, wherein said apparatus further comprises a torso immobilizer coupled to said support surface, wherein said torso immobilizer has an open position and a closed position, and the torso immobilizer encloses at least a portion of the upper body of said person when the torso immobilizer is in said closed position.

17. The method of claim 16 wherein said torso immobilizer comprises:
a pivotably mounted rigid support structure comprising a pair of arms;
armpit supports attached to the pivoted rigid support structure; and
a torso contact surface attached to the pivoted rigid support structure.

18. The apparatus of claim 17, further including providing biasing means attached to said rigid support structure, wherein said biasing means controls the degree to which said arms of said torso immobilizer close.

19. The method of claim 17 wherein said apparatus comprises a contoured support surface interchangeably coupled to said base.

20. The method of claim 19, further comprising
interchanging said support surface to accommodate the body shape of said person.
